(19) United States
(12) Patent Application Publication
(10) Pub. No.: US 2007/0142917 A1
(43) Pub. Date: Jun. 21, 2007

(54) APPARATUS AND METHOD TO OBTAIN BONE FIXATION

(76) Inventors: Christopher P. Roche, Gainesville, FL (US); Pierre Flurin, Bordeaux-Merignac (FR); Thomas Wright, Gainesville, FL (US); Joseph Zuckerman, New York, NY (US)

Correspondence Address: GREENBERG TRAURIG, LLP, MET LIFE BUILDING, 200 PARK AVENUE, NEW YORK, NY, 10166, US

(21) Appl. No.: 11/553,324

(22) Filed: Oct. 26, 2006

Related U.S. Application Data
(60) Provisional application No. 60/730,434, filed on Oct. 26, 2005.

(51) Int. Cl. A61F 2/40; A61F 2/30

(52) U.S. Cl. 623/19.11; 623/23.44; 623/23.43

(57) ABSTRACT

The present invention relates to an apparatus and method to obtain bone fixation. More particularly, one embodiment of the present invention relates to a mechanism for achieving bone “through-growth” in a variety of orthopaedic applications. In one example (which example is intended to be illustrative and not restrictive), a prosthesis designed to achieve bone “through-growth” via one or more “windows” in a peg or keel structure may be provided (bone graft may be inserted into the peg or keel structure). In one specific example (which example is intended to be illustrative and not restrictive), a glenoid prosthesis (and associated method) may be used to resurface the scapula. In another specific example, the present invention may be used in the context of a partial or total shoulder arthroplasty.

### RELATED APPLICATIONS

This application claims the benefit of U.S. Provisional Application Ser. No. 60/730,434, filed Oct. 26, 2005, which is incorporated herein by reference in its entirety.

### FIELD OF THE INVENTION

The present invention relates to an apparatus and method to obtain bone fixation.

More particularly, one embodiment of the present invention relates to a mechanism for achieving bone “through-growth” in a variety of orthopaedic applications.

In one example (which example is intended to be illustrative and not restrictive), a prosthesis designed to achieve bone “through-growth” via one or more “windows” in a peg or keel structure may be provided (bone graft may be inserted into the peg or keel structure).

In one specific example (which example is intended to be illustrative and not restrictive), a glenoid prosthesis (and associated method) may be used to resurface the scapula.

In another specific example (which example is intended to be illustrative and not restrictive), the present invention may be used in the context of a partial or total shoulder arthroplasty.

### BACKGROUND OF THE INVENTION

Various prosthetic devices, for example, shoulder-related prosthetic devices have been proposed. Examples include those disclosed in the following patent publications:

United States Patent Application 20040059424, in the name of Guederian, et al., relates to a metal back prosthetic glenoid component with cemented pegs and hollow metal cage screw. More particularly, this application relates to a prosthetic glenoid component for attachment to a glenoid surface of a scapula to replace a natural socket of a shoulder and to provide a bearing surface for a head portion of an arm bone or humerus. The metal back glenoid component has integrally formed attachment legs which are cemented into corresponding holes formed in the glenoid surface, and also has an opening for receiving a hollow metal cage which is screwed into the glenoid surface.

United States Patent Application 20050209700, in the name of Rockwood, Jr., et al., relates to a method for securing a glenoid component to a scapula. More particularly, this application relates to a medical procedure providing a prosthesis which includes (i) a body portion having a rear surface and a bearing surface, and (ii) an anchor peg including a shaft extending from the rear surface of the body portion, wherein the anchor peg includes a plurality of outwardly extending fins secured to the shaft, and further wherein the plurality of outwardly extending fins each possesses a first diameter. The medical procedure further includes creating an anchor hole in a natural bone, the anchor hole possessing a second diameter which is less than the first diameter. In addition, the method procedure includes positioning the anchor peg within the anchor hole so that each of the plurality of outwardly extending fins deforms so as to possess (i) a concave side which faces an open end of the anchor hole, and (ii) a convex side that faces a closed end of the anchor hole.

United States Patent Application 20050049709, in the name of Tornier, relates to a glenoid component of a shoulder prosthesis and complete shoulder prosthesis incorporating such a component. More particularly, this application relates to a glenoid component comprising a metal body of which the inner face is adapted to be immobilized on the glenoid cavity of a shoulder and of which the outer face bears a concave articulating surface adapted to cooperate with a humeral component. This articulating surface extends on the periphery by a convex surface forming, at least in part, the edge of the body.

United States Patent Application 20050060039, in the name of Cyprien, relates to a shoulder glenoid prosthesis with method and tools for implanting it. More particularly, this application relates to a glenoid plastic prosthesis for cementation, comprising a pear-shaped body having a concave articular face and a convex face with a horizontal keel and threaded pegs. Indentations on the keel and the convex surface of the implant follow a generally fractal pattern in order to increase the implant cement interface. Tools are provided such as a) a glenoid-marking tool used to stamp into the glenoid subchondral bone patterns of indentations of a generally fractal nature and b) a glenoid indentation tool used to make indents in walls of the glenoid cavity for the keel in order to increase the bone-cement interface.

U.S. Pat. No. 6,679,916, in the name of Frankle, et al., relates to a shoulder prosthesis system. More particularly, this patent relates to a shoulder prosthesis system having a glenoid socket with an interior face with couplers and an exterior face being a concave articulating face with a first longitudinal radius of curvature and a second latitudinal radius of curvature. A backing plate has an outer extent and an inner extent. The outer extent has a cylindrical base and a recess around the periphery and a plurality of tapered bores extending through the outer extent spaced between the recess and the center of the backing plate. The inner extent is formed with a projection with threads adapted to be rotatably coupled into a scapula of a patient. The couplers of the outer extent are adapted to snap couple with the recesses of the glenoid socket.

U.S. Pat. No. 6,406,495, in the name of Schoch, relates to a glenoid prosthesis and a modular system with glenoid prostheses. More particularly, this patent relates to a glenoid prosthesis including a bearing shell, the reverse side of which has a plurality of anchoring pins which are arranged to one another. At least one anchoring pin has a coupling element, and at least one sleeve with a fitting securable coupling element and an outer anchoring structure is provided in order to selectively enable a cementing in of the anchoring pin or a mechanical hammering in of the pin together with the sleeve which is fixed to it.

U.S. Pat. No. 6,911,047, in the name of Rockwood, Jr., et al., relates to an apparatus and method for securing a cementless glenoid component to a glenoid surface of a scapula. More particularly, this patent relates to a glenoid component for securement to a glenoid surface of a scapula so as to provide a bearing surface for a head portion of a humerus including a body portion having a first surface configured to contact the glenoid surface of the scapula and a second surface configured to receive the head portion of the humerus. The glenoid component also includes an anchor peg extending from the first surface of the body portion for penetrating the glenoid surface of the scapula so as to secure the body portion to the glenoid surface of the scapula. The anchor peg has a first end portion and a second end portion with the first end portion of the anchor peg being secured to the first surface of the body portion, and the second end portion of the anchor peg having a number of fins secured thereto. The glenoid component also includes a first stabilizing peg extending from the first surface of the body portion for penetrating the glenoid surface of the scapula so as to prevent movement of the body portion relative to the glenoid surface of the scapula. A method of securing a glenoid component to a glenoid surface of a scapula so as to provide a bearing surface for a head portion of a humerus is also disclosed.

U.S. Pat. No. 6,699,289, in the name of lannotti, et al., relates to an augmented glenoid component having an interrupted surface and associated method for securing the augmented glenoid component to a glenoid surface of a scapula. More particularly, this patent relates to a glenoid component for securement to a glenoid surface of a scapula so as to provide a bearing surface for a head portion of a humerus including a body having a first surface configured to contact the glenoid surface of the scapula and a second surface configured to receive the head portion of the humerus. The glenoid component also includes an interruption such as a buttress extending from the body. The interruption is configured to be received in a like-configured notch formed in the glenoid surface of the scapula. The interruption helps prevent movement of the glenoid component relative to the glenoid surface of the scapula after implant. The body also may include anchoring extending from the body for penetrating the glenoid surface of the scapula so as to help secure the body to the glenoid surface of the scapula. The glenoid component is used in one application to help correct bone defects and in another application for wear. A method of securing the glenoid component to a glenoid surface of a scapula so as to provide a bearing surface for a head portion of a humerus is also disclosed.

U.S. Pat. No. 6,514,287, in the name of Ondrla, et al., relates to a modular glenoid assembly having bearing insert. More particularly, this patent relates to a modular glenoid assembly provided for attachment to a glenoid surface of a scapula. The modular glenoid assembly includes a base adapted to couple with the glenoid surface and a bearing insert. The base includes a lip that defines a channel. The bearing insert includes a bearing surface adapted to engage a head portion of a humeral component and a tab. The tab is formed to be received within the channel and engage the lip when the insert is moved in a superior direction into position against the base.

### BRIEF DESCRIPTION OF THE DRAWINGS

FIG. 1A shows a perspective view of one example of the present invention;

FIG. 1B shows a side view of the apparatus of FIG. 1A;

FIG. 1C shows a bottom view of the apparatus of FIG. 1A;

FIG. 2A shows a perspective view of another example of the present invention;

FIG. 2B shows a side view of the apparatus of FIG. 2A;

FIG. 2C shows a bottom view of the apparatus of FIG. 2A;

FIG. 3A shows a perspective view of another example of the present invention;

FIG. 3B shows a side view of the apparatus of FIG. 3A;

FIG. 3C shows a bottom view of the apparatus of FIG. 3A;

FIG. 4A shows a perspective view of another example of the present invention;

FIG. 4B shows a side view of the apparatus of FIG. 4A; and

FIG. 4C shows a bottom view of the apparatus of FIG. 4A.

Among those benefits and improvements that have been disclosed, other objects and advantages of this invention will become apparent from the following description taken in conjunction with the accompanying figures. The figures constitute a part of this specification and include illustrative embodiments of the present invention and illustrate various objects and features thereof.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

One example of the present invention (which example is intended to be illustrative and not restrictive), provides a minimally cemented glenoid prosthesis (and associated method) that conserves glenoid cancellous bone and achieves adequate long-term fixation. In one specific example (which example is intended to be illustrative and not restrictive), this may be accomplished by way of a UHMWPE bearing surface that is molded onto a titanium construct. This construct may be manufactured in such a way that it allows for the insertion of bone graft and allows for bone growth “through” the prosthesis. This bone growth “through” the prosthesis may be in addition to or instead of the bone in-/on-growth that is achieved via surface textures. Of course, rather than (or in combination with) the UHMWPE bearing surface, any other biocompatible bearing surface(s) may be utilized. Further, rather than (or in combination with) the titanium construct, any other biocompatible construct(s) may be utilized.

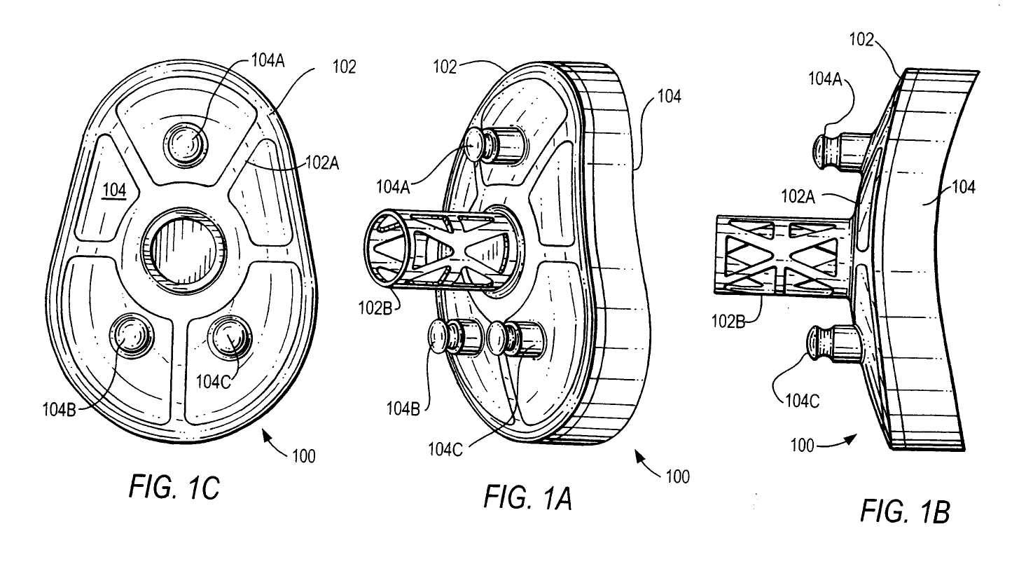

Reference will now be made to FIGS. 1A-C, showing one example of the present invention. As seen in these Figs., Prosthesis 100 may comprise a one piece, molded on metal back, hybrid cemented/cementless glenoid peg design (utilizing a plurality of windows or holes in the peg). More particularly, Metal Construct 102 may comprise Frame Portion 102A and Peg Portion 102B. Further, Bearing Surface 104 (which may be molded-onto Metal Construct 102) may comprise peripheral Protrusions 104A-C (each of which may include one or more grooves). If desired, initial/supplemental fixation may be achieved by securing Protrusions 104A-C to the scapula via cement (the groove(s) may aid in forming a secure bond with the cement). Holes or spaces in the scapula for these Protrusions 104A-C may be prepared with a drill (before and/or after contouring the articular surface). A drill and/or a broach (or similar device(s)) may be used to prepare a hole or space in the scapula for Peg Portion 102B of Metal Construct 102. Graft may be inserted into Peg Portion 102B of Metal Construct 102 (e.g., to replace some or all of the removed cancellous bone) and implanted back into the prepared hole.

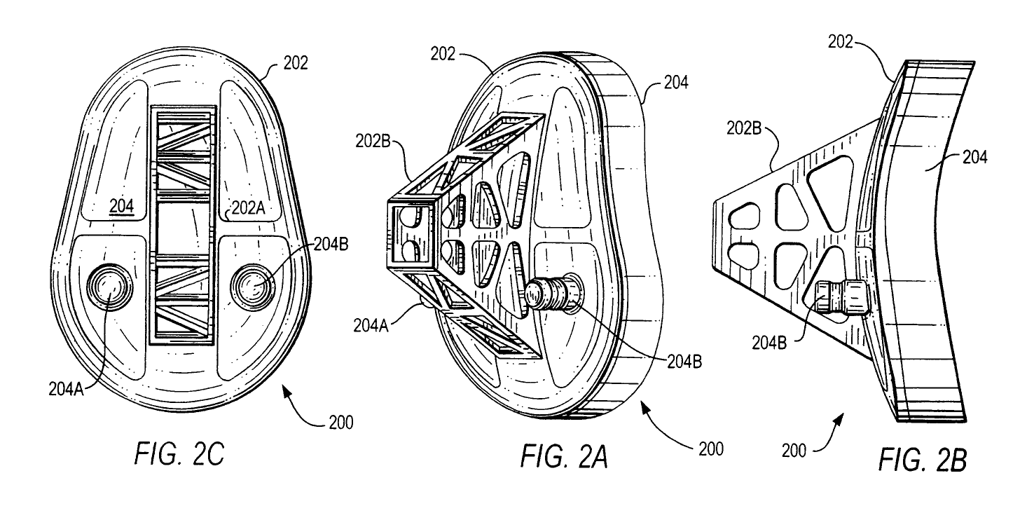

Reference will now be made to FIGS. 2A-C, showing another example of the present invention. As seen in these Figs., Prosthesis 200 may comprise a one piece, molded on metal back, hybrid cemented/cementless windowed keel design (the keel may include a plurality of windows or holes therein). More particularly, Metal Construct 202 may comprise Frame Portion 202A and Keel Portion 202B. Further, Bearing Surface 204 (which may be molded-onto Metal Construct 202) may comprise peripheral Protrusions 204A-B (each of which may include one or more grooves). If desired, initial/supplemental fixation may be achieved by securing Protrusions 204A-B to the scapula via cement (the groove(s) may aid in forming a secure bond with the cement). Holes or spaces in the scapula for these Protrusions 204A-B may be prepared with a drill (before and/or after contouring the articular surface). A drill and/or a broach (or similar device(s)) may be used to prepare a hole or space in the scapula for Keel Portion 202B of Metal Construct 202. Graft may be inserted into Keel Portion 202B of Metal Construct 202 (e.g., to replace some or all of the removed cancellous bone) and implanted back into the prepared hole.

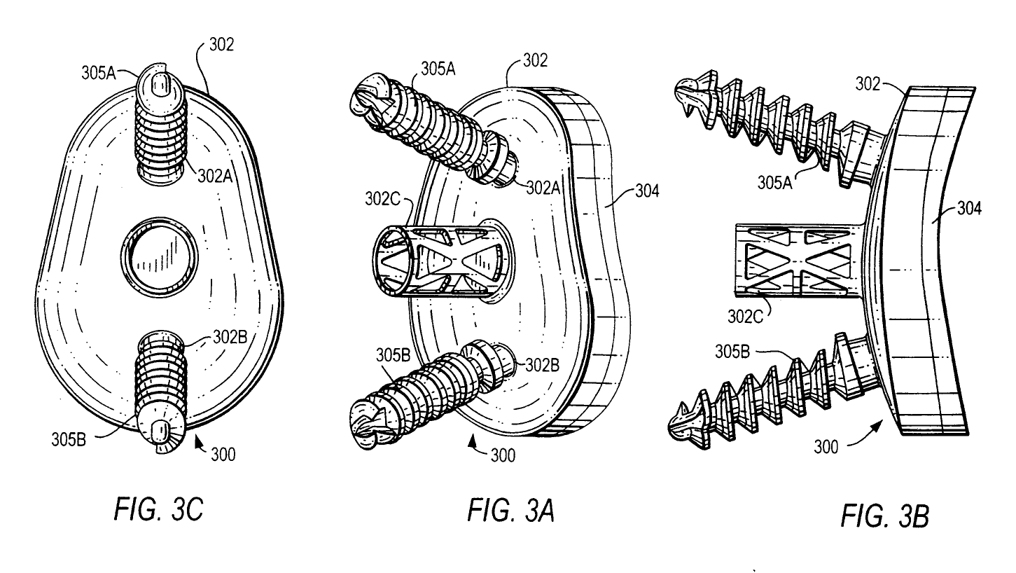

Reference will now be made to FIGS. 3A-C, showing another example of the present invention. As seen in these Figs., Prosthesis 300 may comprise a modular, metal back, glenoid peg design (utilizing a plurality of windows or holes in the peg). More particularly, Metal Construct 302 may comprise Through-Holes 302A, 302B and Peg Portion 302C. Further, Bearing Surface 304 (which may be modular) may be attached in any desired manner to Metal Construct 302. If desired, initial/supplemental fixation may be achieved by securing Screws 305A, 305B to the scapula (thus reducing or eliminating the need for cement). Holes or spaces in the scapula for these Screws 305A, 305B may be prepared with a drill (before and/or after contouring the articular surface). A drill and/or a broach (or similar device(s)) may be used to prepare a hole or space in the scapula for Peg Portion 302C of Metal Construct 302. Graft may be inserted into Peg Portion 302C of Metal Construct 302 (e.g., to replace some or all of the removed cancellous bone) and implanted back into the prepared hole.

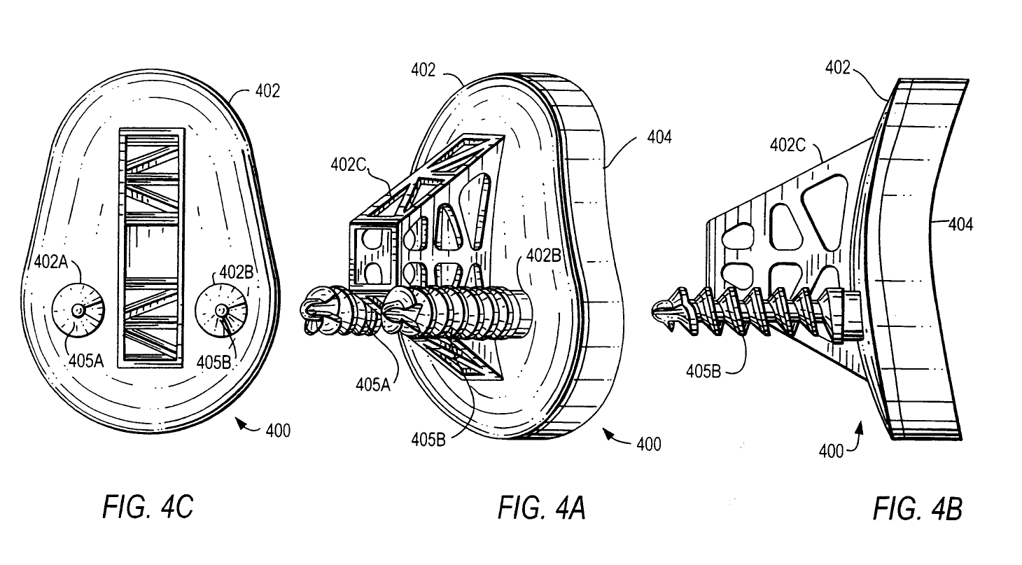

Reference will now be made to FIGS. 4A-C, showing another example of the present invention. As seen in these Figs., Prosthesis 400 may comprise a modular, metal back, windowed keel design (the keel may include a plurality of windows or holes therein). More particularly, Metal Construct 402 may comprise Through-Holes 402A, 402B and Keel Portion 402C. Further, Bearing Surface 404 (which may be modular) may be attached in any desired manner to Metal Construct 402. If desired, initial/supplemental fixation may be achieved by securing Screws 405A, 405B to the scapula (thus reducing or eliminating the need for cement). Holes or spaces in the scapula for these Screws 405A, 405B may be prepared with a drill (before and/or after contouring the articular surface). A drill and/or a broach (or similar device(s)) may be used to prepare a hole or space in the scapula for Keel Portion 402C of Metal Construct 402. Graft may be inserted into Keel Portion 402C of Metal Construct 402 (e.g., to replace some or all of the removed cancellous bone) and implanted back into the prepared hole.

As described above, the present invention may help to achieve, in one example, long-term scapular fixation of the glenoid component without removing a large portion of glenoid cancellous bone stock (of course, long-term fixation of other components to other bones may be provided by the present invention). Additionally, the present invention may provide for the use of a minimum amount of cement (e.g., to reduce thermal-induced bone necrosis and/or operating room time). Moreover, the present invention may provide for the use of a thin bearing component (e.g., to reduce joint stiffness).

Also described above, various embodiments of the present invention may: (a) provide a “windowed” fixation device (e.g., that conserves glenoid cancellous bone stock), (b) induce bone growth and/or provide initial/supplemental fixation. Additionally, various embodiments of the present invention may provide for insertion of bone graft and/or bone “through-growth.” Of course, one or more windows or holes may be utilized - either all of the same size/shape or of varying sizes/shapes (e.g. the prosthesis may utilize multiple size/shape windows or holes ranging from very large to very small).

In another embodiment, the windowed or holed feature (e.g., peg or keel) of the present invention may be designed and manufactured in any desired shape and/or size and can be one or several in number to improve fixation (e.g., based upon availability of glenoid cancellous bone). Further, the windowed or holed feature (e.g., peg or keel) may be placed in the center of the prosthesis, offset from the center of the prosthesis, or both (e.g., the case of multiple features).

In another embodiment, the present invention may be designed and manufactured in such a manner as to maximize the surface area of graft (e.g., for a given volume of bone removed) that contacts the existing bone (e.g., in the scapula). This may thereby increase the probability of native bone in-growth through the graft and reduce the probability of graft resorption.

In another embodiment, the present invention may be manufactured/formed in an integral manner, thereby allowing for a molded on metal design (which, it is believed, is not associated with disassociation and overstuffing).

In another embodiment, the present invention may be used according to traditional partial or total shoulder arthroplasty surgical technique (e.g., reaming, drilling, and broaching) and may not necessarily require a nontraditional surgical technique (e.g., coring reamer).

In another embodiment, the present invention may provide the ability to revise the bearing element (e.g., poly) if need be without disturbing a well-fixed metal back component (particularly in the context of the modular metal-backed example). Of course, in the modular metal-backed example the possibility of liner disassociation and the possibility of over-stuffing the joint due to an increased thickness of the device must be taken into account.

In another embodiment, the present invention may utilize various shapes, sizes, and numbers of windowed features(s) and various shapes, sizes and numbers of doors, windows, holes, and patterns in the windowed features(s) to optimize the insertion of the graft and improve the fixation (e.g., by achieving more reliable “through-growth” of bone).

In another embodiment, the present invention may utilize windowed or holed feature(s) that are designed and manufactured in such a way that one or more of the windowed or holed features are malleable (e.g., thereby allowing the surgeon to conform the construct to the patient's anatomy).

In another embodiment, the present invention may provide for introduction of one or more therapeutic agents (e.g., graft, antibiotics, growth factors) and/or non-therapeutic agents (e.g., cement) into a prepared “bone void” such that the device in its own manner functions as a scaffold or delivery vehicle. In this regard, the device may have a proximal and/or distal opening for insertion of the therapeutic and/or non-therapeutic agents therein.

In another embodiment, a prosthesis for attachment to a bone of a patient is provided, comprising: a metal construct comprising a frame portion with a front and a back and a peg portion extending from the back of the frame portion; and a non-metal bearing surface with a front and a back comprising at least one non-metal protrusion extending from the back of the bearing surface; wherein the bearing surface is attached to the frame portion; wherein the peg portion is configured to be disposed within a first space formed in the bone; and wherein the at least one protrusion is configured to be disposed within a second space formed in the bone.

In one example, the peg portion may have a shape selected from the group consisting of: cylindrical, square, rectangular and elliptical.

In another example, the peg portion may be essentially hollow.

In another example, the peg portion may have a proximal end and a distal end, wherein the proximal end of the peg portion may be attached to the frame portion, and wherein at least one of the proximal end and the distal end may be substantially open.

In another example, the opening in at least one of the proximal end and the distal end may provide access into an interior of the peg portion for placement of at least one of: (a) a therapeutic agent; and (b) a non-therapeutic agent.

In another example, the therapeutic agent may be selected from the group including: (a) at least one supplemental graft material; (b) at least one antibiotic; and (c) at least one growth factor.

In another example, the non-therapeutic agent may comprise cement.

In another example, the peg portion may have a plurality of holes around a perimeter thereof.

In another example, the plurality of holes around the perimeter of the peg portion may provide access into an interior of the peg portion for bone growth from the bone into the peg portion.

In another example, the bearing surface and the at least one protrusion may comprise UHMWPE.

In another example, the bearing surface may be attached to the frame portion by a mechanism selected from the group including: (a) at least one dovetail interface; (b) at least one snap-fit interface; (c) at least one threaded fastener; and (d) being molded-on.

In another example, the bone may be a scapula and the prosthesis may be a glenoid prosthesis.

In another embodiment, a prosthesis for attachment to a bone of a patient may be provided, comprising: a metal construct comprising a frame portion with a front and a back and a keel portion extending from the back of the frame portion; and a non-metal bearing surface with a front and a back comprising at least one non-metal protrusion extending from the back of the bearing surface; wherein the bearing surface is attached to the frame portion; wherein the keel portion is configured to be disposed within a first space formed in the bone; and wherein the at least one protrusion is configured to be disposed within a second space formed in the bone.

In one example, the keel portion may extend in at least one of: (a) an anterior/posterior direction; and (b) a medial/lateral direction.

In another example, the keel portion may be essentially hollow.

In another example, the keel portion may have a proximal end and a distal end, wherein the proximal end of the keel portion may be attached to the frame portion, and wherein at least one of the proximal end and the distal end may be substantially open.

In another example, a height dimension of the keel portion may taper from larger to smaller in a direction moving from the proximal end of the keel portion to the distal end of the keel portion.

In another example, the opening in at least one of the proximal end and the distal end may provide access into an interior of the keel portion for placement of at least one of: (a) a therapeutic agent; and (b) a non-therapeutic agent.

In another example, the therapeutic agent may be selected from the group including: (a) at least one supplemental graft material; (b) at least one antibiotic; and (c) at least one growth factor.

In another example, the non-therapeutic agent may comprise cement.

In another example, the keel portion may have a plurality of holes around a perimeter thereof.

In another example, the plurality of holes around the perimeter of the keel portion may provide access into an interior of the keel portion for bone growth from the bone into the keel portion.

In another example, the bearing surface and the at least one protrusion may comprise UHMWPE.

In another example, the bearing surface may be attached to the frame portion by a mechanism selected from the group including: (a) at least one dovetail interface; (b) at least one snap-fit interface; (c) at least one threaded fastener; and (d) being molded-on.

In another example, the bone may be a scapula and the prosthesis may be a glenoid prosthesis.

In another embodiment, a prosthesis for attachment to a bone of a patient is provided, comprising: a metal construct comprising a frame portion with a front and a back and a keel portion extending from the back of the frame portion; and a non-metal bearing surface with a front and a back; wherein the bearing surface is attached to the frame portion; wherein the keel portion is configured to be disposed within a space formed in the bone; wherein the keel portion has a proximal end and a distal end, wherein the proximal end of the keel portion is attached to the frame portion, and wherein at least one of the proximal end and the distal end is substantially open; wherein the opening in at least one of the proximal end and the distal end provides access into an interior of the keel portion for placement of at least one of: (a) a therapeutic agent; and (b) a non-therapeutic agent; and wherein the keel portion has a shape which is non-cylindrical.

In another example, the keel portion may be essentially hollow.

In another example, the keel portion may extend in at least one of: (a) an anterior/posterior direction; and (b) a medial/lateral direction.

In another example, a height dimension of the keel portion may taper from larger to smaller in a direction moving from the proximal end of the keel portion to the distal end of the keel portion.

In another example, the therapeutic agent may be selected from the group including: (a) at least one supplemental graft material; (b) at least one antibiotic; and (c) at least one growth factor.

In another example, the non-therapeutic agent may comprise cement.

In another example, the keel portion may have a plurality of holes around a perimeter thereof.

In another example, the plurality of holes around the perimeter of the keel portion may provide access into an interior of the keel portion for bone growth from the bone into the keel portion.

In another example, the bearing surface may comprise UHMWPE.

In another example, the bearing surface may be attached to the frame portion by a mechanism selected from the group including: (a) at least one dovetail interface; (b) at least one snap-fit interface; (c) at least one threaded fastener; and (d) being molded-on.

In another example, the bone may be a scapula and the prosthesis may be a glenoid prosthesis.

In another embodiment, a method for attaching a prosthesis to a bone of a patient is provided, comprising: forming a space in the bone; inserting a keel portion of a metal construct into the space formed in the bone, wherein the metal construct comprises a frame portion with a front and a back and the keel portion extends from the back of the frame portion; and inserting into an interior of the keel portion through an opening in a proximal end of the keel portion at least one of: (a) a therapeutic agent; and (b) a non-therapeutic agent; wherein the insertion into the interior of the keel portion through the opening in the proximal end of the keel portion of at least one of: (a) the therapeutic agent; and (b) the non-therapeutic agent is carried out after the keel portion is inserted into the space formed in the bone.

In one example, the keel portion may have a shape which is non-cylindrical.

In another example, the therapeutic agent may be selected from the group including: (a) at least one supplemental graft material; (b) at least one antibiotic; and (c) at least one growth factor.

In another example, the non-therapeutic agent may comprise cement.

In another example, the method may further comprise attaching a non-metal bearing surface to the frame portion after the insertion into the interior of the keel portion through the opening in the proximal end of the keel portion at least one of: (a) the therapeutic agent; and (b) the non-therapeutic agent.

In another example, the bearing surface may be attached to the frame portion by a mechanism selected from the group including: (a) at least one dovetail interface; (b) at least one snap-fit interface; and (c) at least one threaded fastener.

In another example, the bone may be a scapula and the prosthesis may be a glenoid prosthesis.

In another example, the peg portion may extend from the frame portion in a generally perpendicular direction.

In another example, the peg portion may extend from the frame portion at an angle other than 90 degrees.

In another example, the at least one protrusion may have a generally cylindrical shape.

In another example, the at least one protrusion may comprise at least one groove around a perimeter thereof.

In another example, the prosthesis may comprise a plurality of protrusions extending from the back of the bearing surface.

In another example, the frame portion and the peg portion may be formed from the same material.

In another example, the frame portion and the peg portion may be formed from different materials.

In another example, the peg portion may be malleable.

In another example, the frame portion and the keel portion may be formed from the same material.

In another example, the frame portion and the keel portion may be formed from different materials.

In another example, the keel portion may be malleable.

While a number of embodiments of the present invention have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from any of the following materials: (a) any biocompatible material (which biocompatible material may be treated to permit surface bone ingrowth or prohibit surface bone ingrowth—depending upon the desire of the surgeon); (b) a plastic; (c) a fiber; (d) a polymer; (e) a metal (a pure metal such as titanium and/or an alloy such as Ti-Al-Nb, Ti-6Al-4V, stainless steel); (f) any combination thereof. Further still, the metal construct may be a machined metal construct. Further still, various Peg designs (e.g. square/elliptical/angled pegs) may be utilized. Further still, various Keel designs (e.g. anterior/posterior keel, medial/lateral keel, dorsal fin keel, angled keel) may be utilized. Further still, the prosthesis may utilize one or more modular elements. Further still, any desired number of peg(s) and/or keel(s) may be utilized with a given prosthesis. Further still, any number of protrusions (e.g., such as for initial fixation by forming a bond with cement and/or such as for supplemental fixation by forming a bond with cement) may be utilized with a given prosthesis. Further still, any number of female features that increase the cement mantle may be utilized with a given prosthesis. Further still, any number of male features that could dig into the bone so that initial/supplemental fixation can be improved may be utilized with a given prosthesis. Further still, any number of bone screws (e.g., such as for initial fixation and/or such as for supplemental fixation) may be utilized with a given prosthesis. Further still, a bearing surface (e.g., a poly bearing surface) may be attached (e.g., molded) directly to the backside of the peg or keel cage (that is, without a metal construct (e.g., skeleton-type metal construct) between the bearing surface and the peg or keel cage). Further still, the bearing surface protrusion(s) may be separate components that are attached to the bearing surface and/or may be formed as an integral part of the bearing surface. Further still, the bearing surface protrusion(s) may be flexible (e.g., to aid the surgeon in placement of the prosthesis in the body). Further still, any steps described herein may be carried out in any desired order (and any additional steps may be added as desired and any steps may be deleted as desired).

What is claimed is:

1. A prosthesis for attachment to a bone of a patient, comprising:
a metal construct comprising a frame portion with a front and a back and a peg portion extending from the back of the frame portion; and
a non-metal bearing surface with a front and a back comprising at least one non-metal protrusion extending from the back of the bearing surface;
wherein the bearing surface is attached to the frame portion;
wherein the peg portion is configured to be disposed within a first space formed in the bone; and
wherein the at least one protrusion is configured to be disposed within a second space formed in the bone.

2. The prosthesis of claim 1, wherein the peg portion has a shape selected from the group consisting of: cylindrical, square, rectangular and elliptical.

3. The prosthesis of claim 1, wherein the peg portion is essentially hollow.

4. The prosthesis of claim 3, wherein the peg portion has a proximal end and a distal end, wherein the proximal end of the peg portion is attached to the frame portion, and wherein at least one of the proximal end and the distal end is substantially open.

5. The prosthesis of claim 4, wherein the opening in at least one of the proximal end and the distal end provides access into an interior of the peg portion for placement of at least one of: (a) a therapeutic agent; and (b) a non-therapeutic agent.

6. The prosthesis of claim 5, wherein the therapeutic agent is selected from the group consisting of: (a) at least one supplemental graft material; (b) at least one antibiotic; and (c) at least one growth factor.

7. The prosthesis of claim 5, wherein the non-therapeutic agent comprises cement.

8. The prosthesis of claim 2, wherein the peg portion has a plurality of holes around a perimeter thereof.

9. The prosthesis of claim 8, wherein the plurality of holes around the perimeter of the peg portion provide access into an interior of the peg portion for bone growth from the bone into the peg portion.

10. The prosthesis of claim 1, wherein the bearing surface and the at least one protrusion comprise UHMWPE.

11. The prosthesis of claim 1, wherein the bearing surface is attached to the frame portion by a mechanism selected from the group consisting of: (a) at least one dovetail interface; (b) at least one snap-fit interface; (c) at least one threaded fastener; and (d) being molded-on.

12. The prosthesis of claim 1, wherein the bone is a scapula and the prosthesis is a glenoid prosthesis.

13. A prosthesis for attachment to a bone of a patient, comprising:
a metal construct comprising a frame portion with a front and a back and a keel portion extending from the back of the frame portion; and
a non-metal bearing surface with a front and a back comprising at least one non-metal protrusion extending from the back of the bearing surface;
wherein the bearing surface is attached to the frame portion;
wherein the keel portion is configured to be disposed within a first space formed in the bone; and
wherein the at least one protrusion is configured to be disposed within a second space formed in the bone.

14. The prosthesis of claim 13, wherein the keel portion extends in at least one of: (a) an anterior/posterior direction; and (b) a medial/lateral direction.

15. The prosthesis of claim 13, wherein the keel portion is essentially hollow.

16. The prosthesis of claim 15, wherein the keel portion has a proximal end and a distal end, wherein the proximal end of the keel portion is attached to the frame portion, and wherein at least one of the proximal end and the distal end is substantially open.

17. The prosthesis of claim 16, wherein a height dimension of the keel portion tapers from larger to smaller in a direction moving from the proximal end of the keel portion to the distal end of the keel portion.

18. The prosthesis of claim 16, wherein the opening in at least one of the proximal end and the distal end provides access into an interior of the keel portion for placement of at least one of: (a) a therapeutic agent; and (b) a non-therapeutic agent.

19. The prosthesis of claim 18, wherein the therapeutic agent is selected from the group consisting of: (a) at least one supplemental graft material; (b) at least one antibiotic; and (c) at least one growth factor.

20. The prosthesis of claim 18, wherein the non-therapeutic agent comprises cement.

21. The prosthesis of claim 13, wherein the keel portion has a plurality of holes around a perimeter thereof.

22. The prosthesis of claim 21, wherein the plurality of holes around the perimeter of the keel portion provide access into an interior of the keel portion for bone growth from the bone into the keel portion.

23. The prosthesis of claim 13, wherein the bearing surface and the at least one protrusion comprise UHMWPE.

24. The prosthesis of claim 13, wherein the bearing surface is attached to the frame portion by a mechanism selected from the group consisting of: (a) at least one dovetail interface; (b) at least one snap-fit interface; (c) at least one threaded fastener; and (d) being molded-on.

25. The prosthesis of claim 13, wherein the bone is a scapula and the prosthesis is a glenoid prosthesis.

26. A prosthesis for attachment to a bone of a patient, comprising:
a metal construct comprising a frame portion with a front and a back and a keel portion extending from the back of the frame portion; and
a non-metal bearing surface with a front and a back;
wherein the bearing surface is attached to the frame portion;
wherein the keel portion is configured to be disposed within a space formed in the bone;
wherein the keel portion has a proximal end and a distal end, wherein the proximal end of the keel portion is attached to the frame portion, and wherein at least one of the proximal end and the distal end is substantially open;
wherein the opening in at least one of the proximal end and the distal end provides access into an interior of the keel portion for placement of at least one of: (a) a therapeutic agent; and (b) a non-therapeutic agent; and
wherein the keel portion has a shape which is non-cylindrical.

27. The prosthesis of claim 26, wherein the keel portion is essentially hollow.

28. The prosthesis of claim 26, wherein the keel portion extends in at least one of: (a) an anterior/posterior direction; and (b) a medial/lateral direction.

29. The prosthesis of claim 26, wherein a height dimension of the keel portion tapers from larger to smaller in a direction moving from the proximal end of the keel portion to the distal end of the keel portion.

30. The prosthesis of claim 26, wherein the therapeutic agent is selected from the group consisting of: (a) at least one supplemental graft material; (b) at least one antibiotic; and (c) at least one growth factor.

31. The prosthesis of claim 26, wherein the non-therapeutic agent comprises cement.

32. The prosthesis of claim 26, wherein the keel portion has a plurality of holes around a perimeter thereof.

33. The prosthesis of claim 32, wherein the plurality of holes around the perimeter of the keel portion provide access into an interior of the keel portion for bone growth from the bone into the keel portion.

34. The prosthesis of claim 26, wherein the bearing surface comprises UHMWPE.

35. The prosthesis of claim 26, wherein the bearing surface is attached to the frame portion by a mechanism selected from the group consisting of: (a) at least one dovetail interface; (b) at least one snap-fit interface; (c) at least one threaded fastener; and (d) being molded-on.

36. The prosthesis of claim 26, wherein the bone is a scapula and the prosthesis is a glenoid prosthesis.

37. A method for attaching a prosthesis to a bone of a patient, comprising:
forming a space in the bone;
inserting a keel portion of a metal construct into the space formed in the bone, wherein the metal construct comprises a frame portion with a front and a back and the keel portion extends from the back of the frame portion; and
inserting into an interior of the keel portion through an opening in a proximal end of the keel portion at least one of: (a) a therapeutic agent; and (b) a non-therapeutic agent;
wherein the insertion into the interior of the keel portion through the opening in the proximal end of the keel portion of at least one of: (a) the therapeutic agent; and (b) the non-therapeutic agent is carried out after the keel portion is inserted into the space formed in the bone.

38. The method of claim 37, wherein the keel portion has a shape which is non-cylindrical.

39. The method of claim 37, wherein the therapeutic agent is selected from the group consisting of: (a) at least one supplemental graft material; (b) at least one antibiotic; and (c) at least one growth factor.

40. The method of claim 37, wherein the non-therapeutic agent comprises cement.

41. The method of claim 37, further comprising attaching a non-metal bearing surface to the frame portion after the insertion into the interior of the keel portion through the opening in the proximal end of the keel portion at least one of: (a) the therapeutic agent; and (b) the non-therapeutic agent.

42. The method of claim 41, wherein the bearing surface is attached to the frame portion by a mechanism selected from the group consisting of: (a) at least one dovetail interface; (b) at least one snap-fit interface; and (c) at least one threaded fastener.

43. The method of claim 37, wherein the bone is a scapula and the prosthesis is a glenoid prosthesis.
